(19) United States
(12) Patent Application Publication
(10) Pub. No.: US 2009/0093827 A1
(43) Pub. Date: Apr. 9, 2009

(54) SUBCUTANEOUS WAIST BAND AND METHODS RELATED THERETO

(75) Inventors: James F. Zucherman, San Francisco, CA (US); Ken Y. Hsu, San Francisco, CA (US)

Correspondence Address: FLIESLER MEYER LLP, 650 CALIFORNIA STREET, 14TH FLOOR, SAN FRANCISCO, CA, 94108, US

(73) Assignee: SPARTEK MEDICAL, INC., Alameda, CA (US)

(21) Appl. No.: 12/237,053

(22) Filed: Sep. 24, 2008

Related U.S. Application Data
(60) Provisional application No. 60/977,978, filed on Oct. 5, 2007.

(51) Int. Cl. A61B 17/12

(52) U.S. Cl. 606/151; 128/898

(57) ABSTRACT

A subcutaneously placed abdominal band for waistline reduction and methods related thereto, wherein an abdominal band is subcutaneously wrapped around a person's waistline underneath the subcutaneous fat and adjacent to a facia and a muscle, the abdominal band being tightened and secured to the body thereby tightening the person's abdominal wall and reducing the person's waistline.

### CLAIM OF PRIORITY

This application claims priority to U.S. Provisional Application No. 60/977,978, filed Oct. 5, 2007, entitled “Subcutaneous Waist Band and Methods Related Thereto” (Attorney Docket No. SPART-01033US0), which is incorporated herein by reference in its entirety.

### FIELD OF THE INVENTION

The present invention generally relates to a subcutaneously placed abdominal band for waistline reduction and methods related thereto.

### BACKGROUND

Numerous methods currently exist to reduce the appearance of a protruding abdomen. These methods can range from using a device to externally constrict or tighten the abdomen (such as a corset) to undergoing a major surgical procedure to alter the shape of the abdominal wall. These methods, however, have their drawbacks. For example, a person may wish to tighten his or her abdominal region in a manner which is inconspicuous, even when the person is unclothed. The use of a corset or any other exterior device to constrict the abdomen would obviously be unsuitable. The person may elect to undergo an abdominoplasty or “tummy tuck” which is a cosmetic surgery procedure used to tighten and narrow the abdominal wall. Nevertheless, a complete abdominoplasty can involve the following steps: making an incision from hip to hip just above the pubic area, making another incision to free the navel from the surrounding skin, detaching the skin from the abdominal wall from an area around the hips all the way up to the ribs (as shown in FIG. 1A), stretching the underlying muscle fascia wall and stitching it into place in its new position (as shown in FIG. 1B), tightening the remaining skin and removing the excess fat, and stitching the skin to close the wound. As is readily apparent, an abdominoplasty may be too invasive for many individuals. Accordingly, what is needed is a method to tighten the muscle and fascia of the abdominal wall that does not rely on any external devices and does not involve highly invasive surgical procedures.

### BRIEF DESCRIPTION OF THE DRAWINGS

The accompanying drawings, which are incorporated into and constitute a part of this specification, illustrate one or more embodiments and, together with the detailed description, serve to explain the principles and implementations of the invention. In the drawings:

FIGS. 1A and 1B are front views of a human torso illustrating the operative techniques used during a typical abdominoplasty in the prior art.

FIG. 2A is a rear view of a human torso illustrating an incision in the skin.

FIG. 2B is a front view of a human torso illustrating two incisions in the skin.

FIG. 2C is a side view of a human torso illustrating an incision in the skin.

FIG. 2D is a front view of a human torso illustrating a peri-umbilical incision in the skin curved cephalad to the umbilicus.

FIG. 3 is a cross-sectional view of the skin, fat, fascia and muscle along an abdominal wall.

FIG. 4 is a cross-sectional view of the skin, fat, fascia and muscle along an abdominal wall, wherein dissection of a plane of dissection adjacent to the abdominal wall has been initiated.

FIG. 5A is a rear view of a human torso illustrating the use of a curved probe to dilate the plane of dissection.

FIG. 5B is a front view of a human torso illustrating the use of a curved probe to dilate the plane of dissection.

FIG. 5C is a side view of a human torso illustrating the use of a curved probe to dilate the plane of dissection.

FIG. 6A illustrates a perspective view of a human torso wherein a plane of dissection has been created around the abdomen below the umbilicus.

FIG. 6B illustrates a perspective view of a human torso wherein a plane of dissection has been created around the abdomen above the umbilicus.

FIG. 6C illustrates a perspective view of a human torso wherein a plane of dissection has been created around the abdomen above and below the umbilicus.

FIG. 7A illustrates a rear view of a human torso wherein a band is being implanted subcutaneously for waistline reduction.

FIG. 7B illustrates a side view of a human torso wherein a band has been implanted subcutaneously for waistline reduction.

FIG. 8A illustrates an embodiment of a band to be implanted around an abdomen for waistline reduction.

FIGS. 8B-8N illustrate different embodiments of fasteners used on a band to be implanted around an abdomen for waistline reduction.

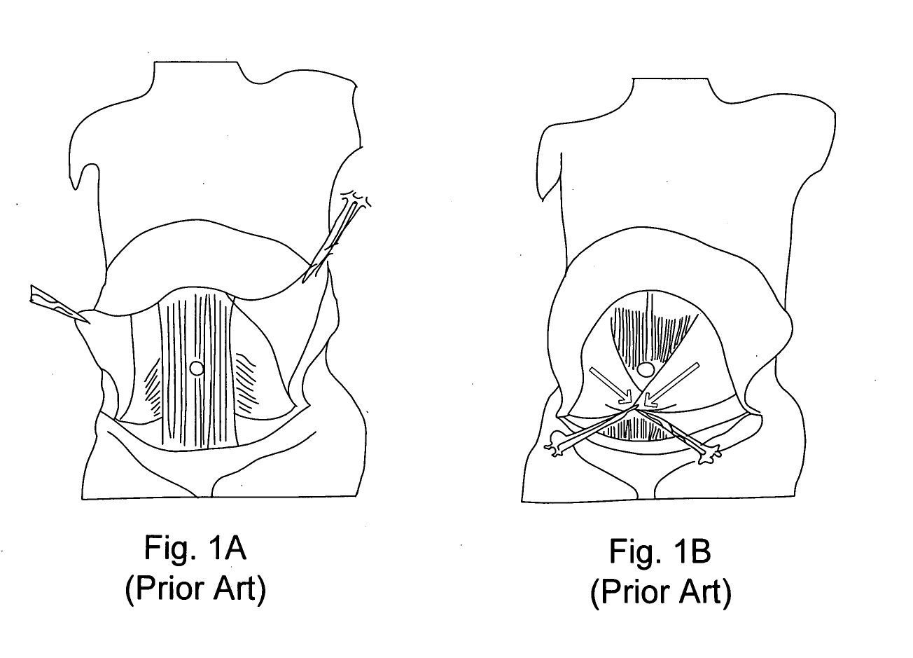

### DETAILED DESCRIPTION

Embodiments are described herein in the context of a subcutaneously implanted abdominal band for waistline reduction and methods related thereto. Those of ordinary skill in the art will realize that the following detailed description is illustrative only and is not intended to be in any way limiting. Other embodiments of the present invention will readily suggest themselves to such skilled persons having the benefit of this disclosure. Reference will now be made in detail to implementations of embodiment of the present invention as illustrated in the accompanying drawings. The same reference indicators will be used throughout the drawings and the following detailed description to refer to the same or like parts.

One of the objects of the invention is to provide a band (or “belt”) which can be surgically implanted subcutaneously to tighten the muscle and fascia of the abdominal wall. Another object of the invention is to provide a minimally invasive method of tightening the muscle and fascia of the abdominal wall using the above mentioned subcutaneous band. Other objects of the invention will become apparent from time to time throughout the specification and claims as hereinafter related.

These and other objects of the invention can be accomplished by surgically implanting a band subcutaneously around a person's waist (or “midline”) to tighten the muscle and fascia of the abdominal wall. The method of implanting the band can briefly be described as follows. First, at least one incision is made to a predetermined location around a person's waist, the incision extending through the subcutaneous fat and superficial to the fascia and muscle. Next a plane of dissection (or “plane” or “path”) is developed around the person's waist underneath the point or points of incision. The plane is developed adjacent to the fascia and muscle tissue layer wherein the fat layer is separated from the fascia and muscle tissue. Once the plane is developed, a first end of a band is inserted into the plane through an incision and wrapped around the person's waist until the first end of the band reaches the original insertion point. The first end of the band can then be withdrawn from the plane of dissection through the incision. The band can then be tightened and tied anteriorly. Finally, the band is subcutaneously secured to the body adjacent the facia and muscle whereby the abdominal wall is tightened by the band.

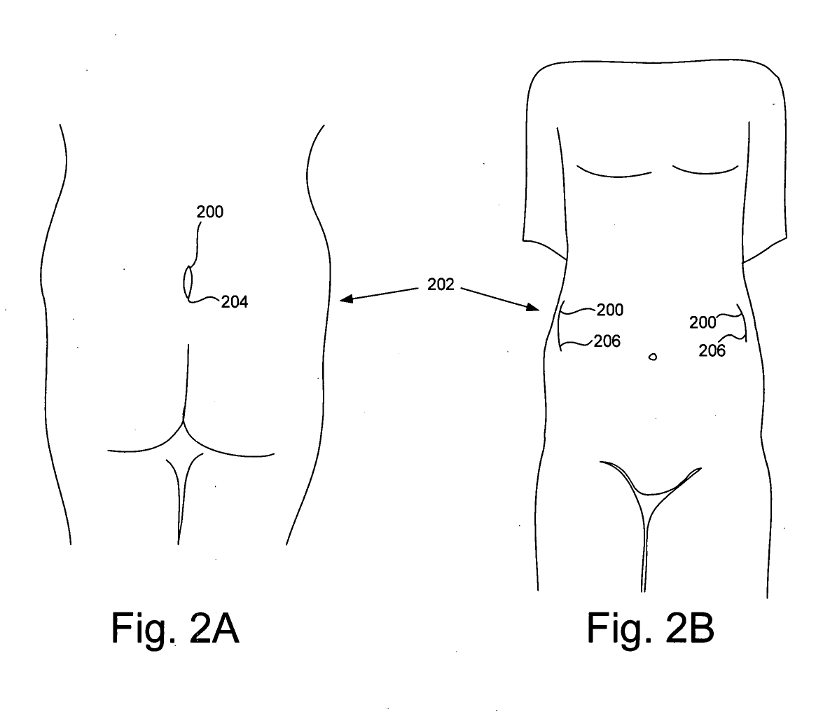

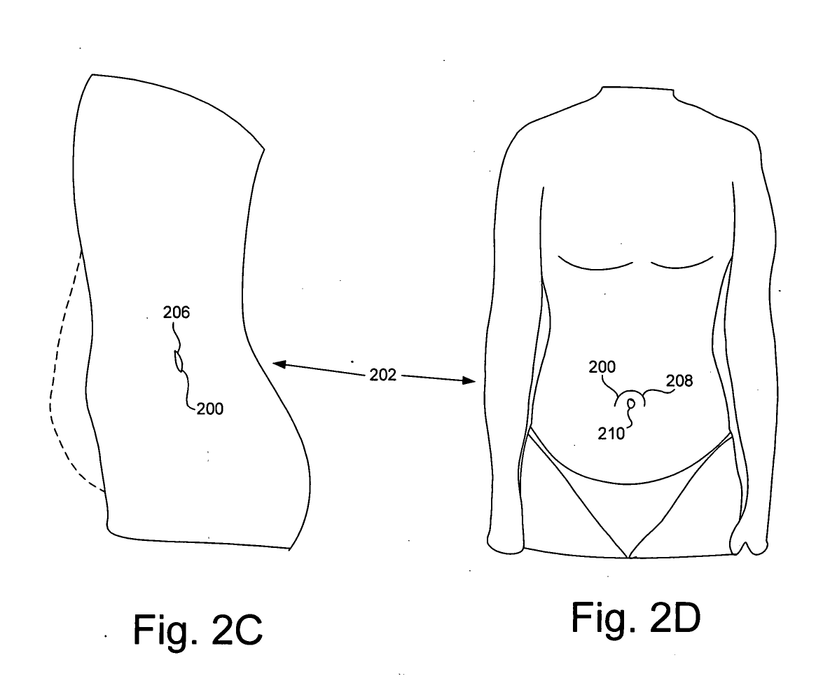

The method of implanting the band will now be described in greater detail. Referring now to FIGS. 2A, 2B, 2C and 2D, the method is commenced by making one or more incisions 200 to the person's midline 202 at predetermined locations. In an embodiment, three skin incisions 200 can be made along the person's midline 202, one incision 200 being a posterior 204 incision 200 (as shown in FIG. 2A) and the other two incisions 200 being lateral 206 incisions 200 (as shown in FIGS. 2B and 2C). In an embodiment, a peri-umbilical 208 incision 200 curved cephalad to the umbilicus 210 (as shown in FIG. 2D) may be made in addition to, or in lieu of, the posterior 204 and lateral 206 incisions 200. The length of the incisions 200 can vary depending on the width of the band being implanted in the patent. In an embodiment, the length of the incision 200 can range from one to three inches. It is noted that any number of incisions having any length may be made to the person without deviating from the scope of this invention.

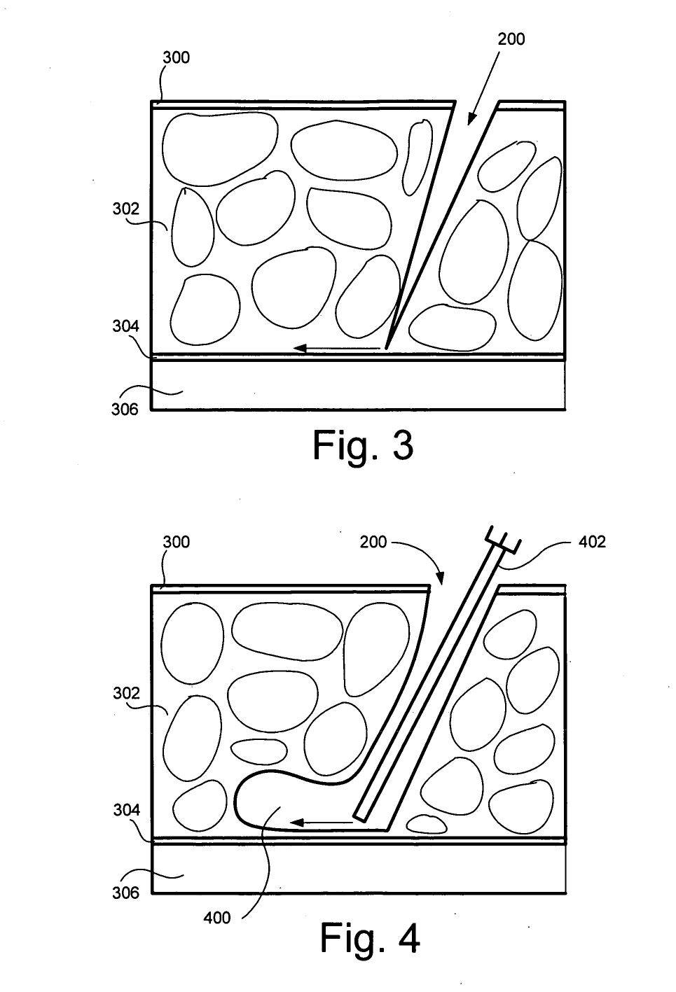

Referring to FIG. 3, the incision 200 is made through the skin 300 and underlying subcutaneous fat 302 and superficial to the fascia 304 and muscle 306. It is preferable that the incision 200 extend completely through the subcutaneous fat 302 to the fascia 304 and muscle 306. This may be accomplished by making an initial incision 200 with a scalpel and then cutting the subcutaneous fat 302 with dissecting scissors until a path to the fascia 304 and muscle 306 is created.

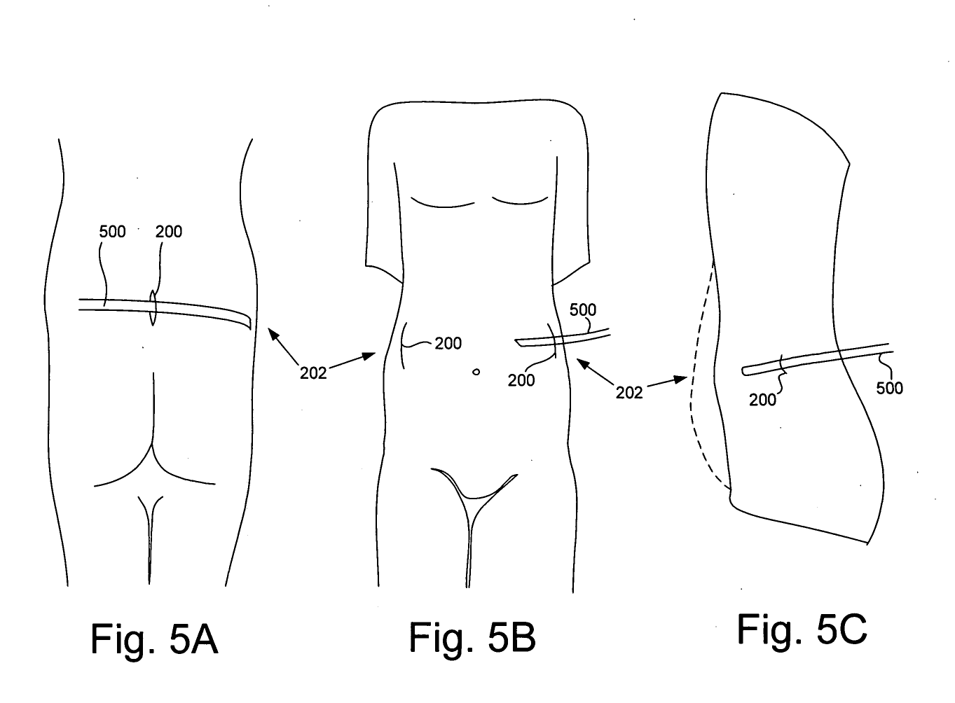

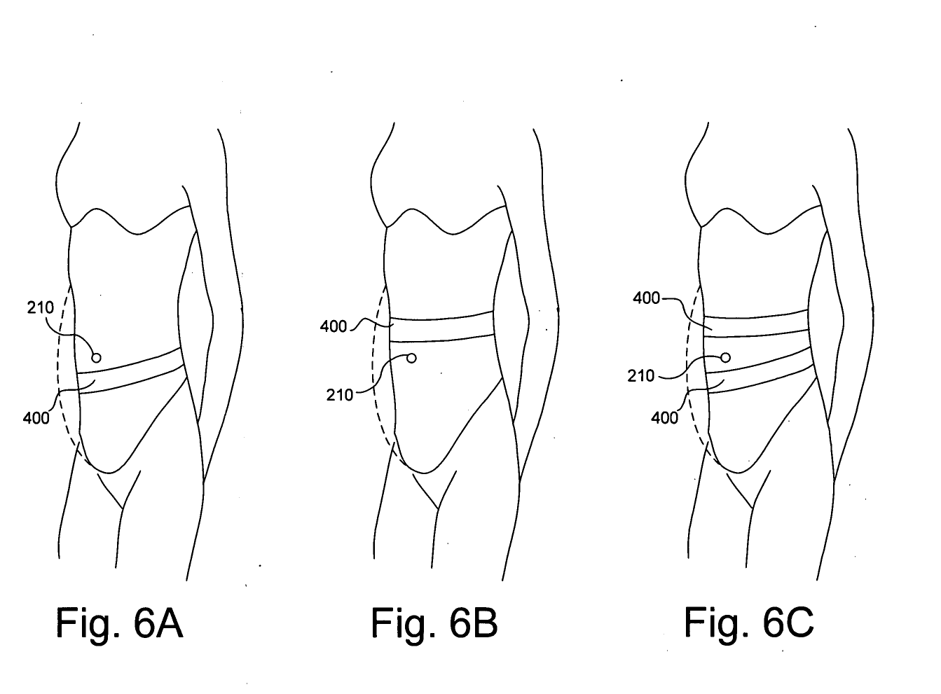

Referring to FIG. 4, after the one or more incisions 200 are made, a plane of dissection 400 can be developed deep to the subcutaneous fat 302 and superficial to the fascia 304 and muscle 306 (shown in FIG. 3) around the person's midline 202 (shown in FIG. 2A). Since fat trapped under pressure can necrose, the plane of dissection 400 is preferably located beneath all subcutaneous fat 302 adjacent to the fascia 304 and muscle 306. In an embodiment, a tool for dissection 402 is inserted into an incision 200 and advanced alongside the person's midline 202 (shown in FIG. 2A) to dissect the structure along the plane of dissection 400. In an embodiment, endoscopic dissection methods can be used. The endoscopic dissection methods can include, but are not limited to, hydro dissection, balloon dissection, ultrasonic dissection, and laser dissection may be employed to dissect the structure along the plane of dissection 400. Endoscopic dissection allows the surgeon to accurately visualize the correct plane of dissection 400 adjacent to the surface of fascia 304 and muscle 306 to ensure that it is devoid of subcutaneous fat 302. As illustrated in FIGS. 5A, 5B and 5C, a curved probe 500 may also be inserted into the one or more incisions 200 along the plane of dissection 400 (not shown) to dilate the path. A surgeon may wish to use smaller probes 500 initially, followed by larger probes 500 during this process. In an embodiment, the plane of dissection 400 can be made below the umbilicus 210 as illustrated in FIG. 6A. In another embodiment, the plane of dissection 400 can be made above the umbilicus 210 as illustrated in FIG. 6B. In yet another embodiment, the plane of dissection 400 can be made both below and above the umbilicus 210 as illustrated in FIG. 6C.

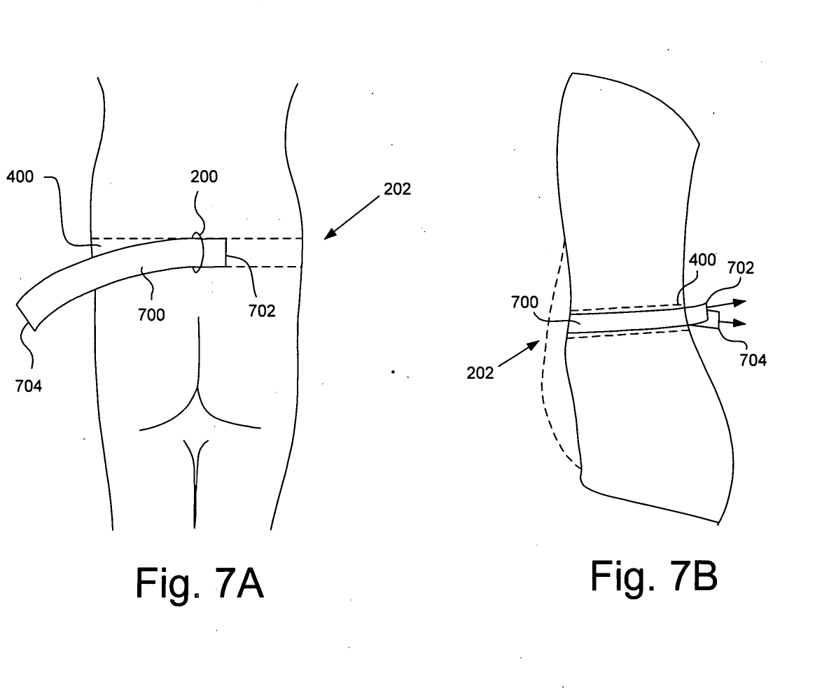

Referring to FIG. 7A, once the plane of dissection 400 has been defined, a band 700 including a first end 702 and a second end 704 may be implanted into the person. When implanting the band 700, the first end 702 of the band is passed through an incision 200 and into the plane of dissection 400. The first end 702 of the band 700 is wrapped around the person's midsection 202 within the plane of dissection 400 until the first end 702 of the band 700 reaches the original insertion point. The first end 702 of the band 700 can then removed from the plane of dissection 400 as shown in FIG. 6B. Once the first end 702 of the band 700 is removed from the plane of dissection 400, the band 700 can be anteriorily tightened and the two ends 702, 704 of the band 700 can be fastened, tied, clipped, clamped and/or otherwise connected to each other to secure the band 700 to the body. In an embodiment, the band 700 can be pulled through the plane of dissection 400 by pulling on a wire, suture or strap attached to the first end 702 of the band 700. In another embodiment, the band 700 is pushed through the plane of dissection 400. The band 700 can be pushed or pulled using a suture passer. Once the band 700 is tightened and tied, the band 700 is secured to the body and the incisions are stitched closed while the band remains wrapped around the person's abdomen.

Different embodiments for the band 700 generally described above can also be utilized. The band 700 is preferably 20 to 30 inches long (generally 20 to 40 or 50 inches long), preferably at least 1.25 inches wide (generally 1 inch to 5 inches wide or of varying width around the length as for example in the shape of a cummerbund) and preferably 1 to 3 mm thick. The band 700 is preferably constructed of a material which is biocompatible, nonresorbable and nonbiodegradable. Specific materials which may be used to construct the band 700 include, but are not limited to, dacroncrystalline polypropylene, polyethylene, polyester fiber, PLLA, PDLA, polyurethane, nylon, titanium mesh, silicon, silastic and other polymers. In an embodiment the band 700 can be elastic and/or reversible.

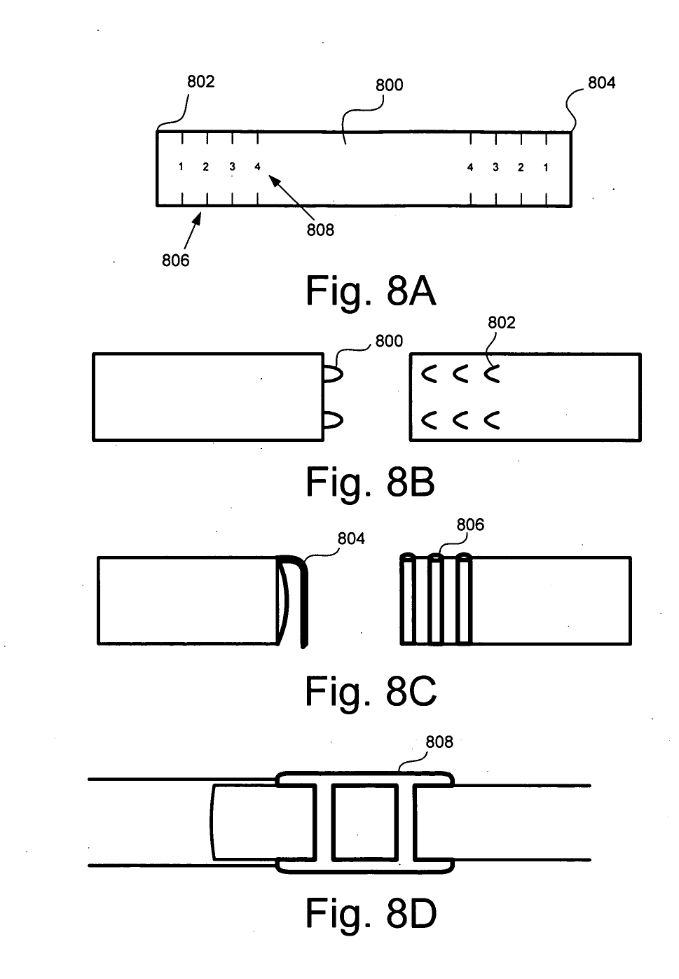

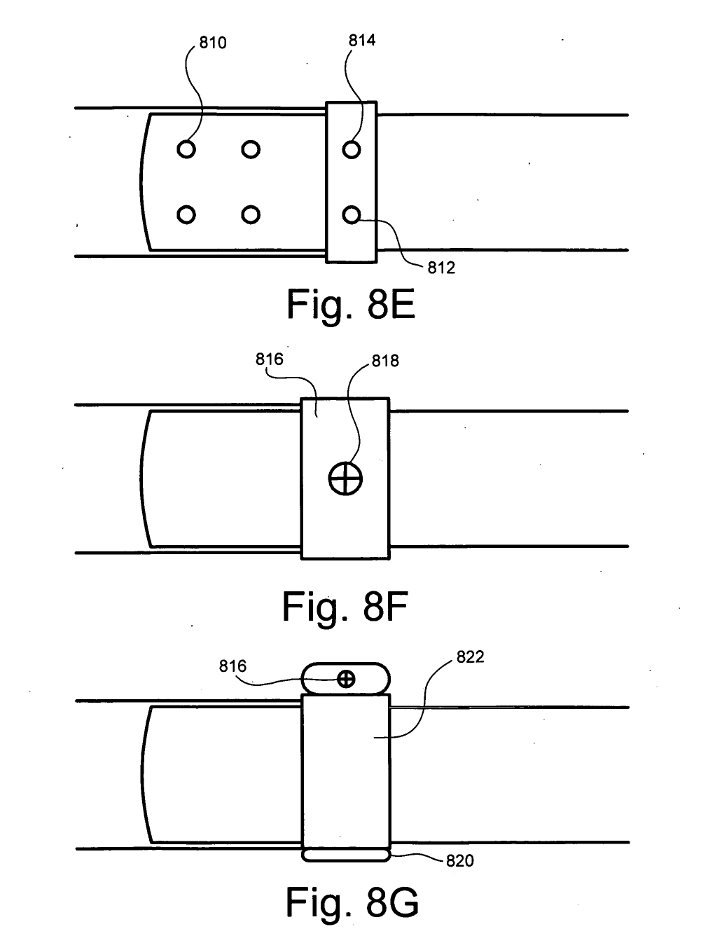

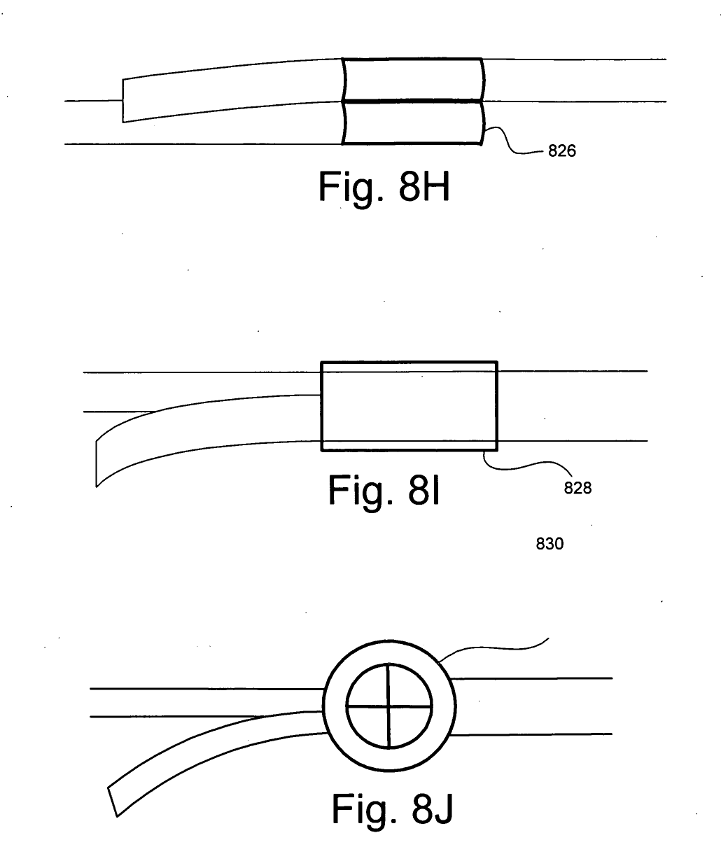

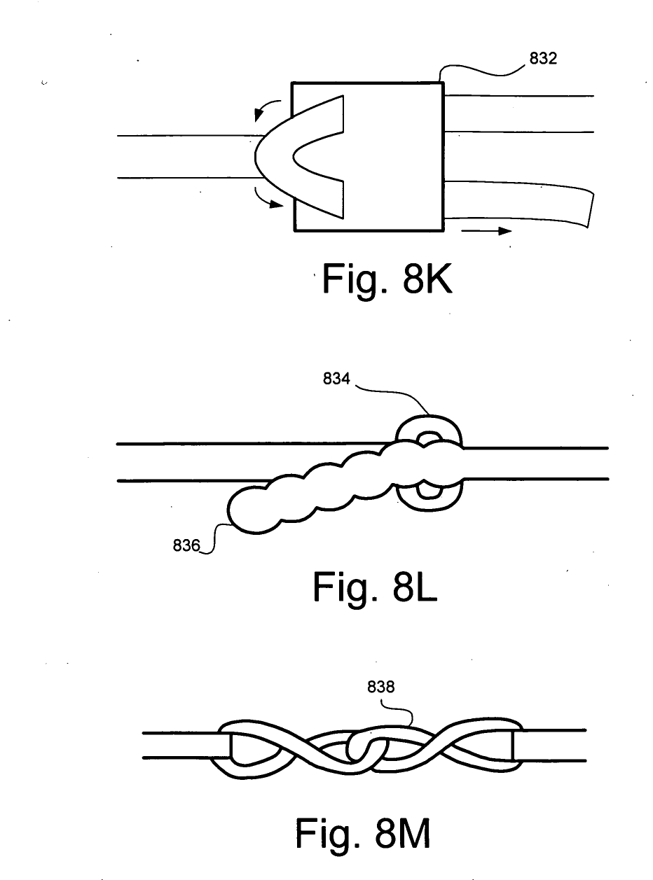

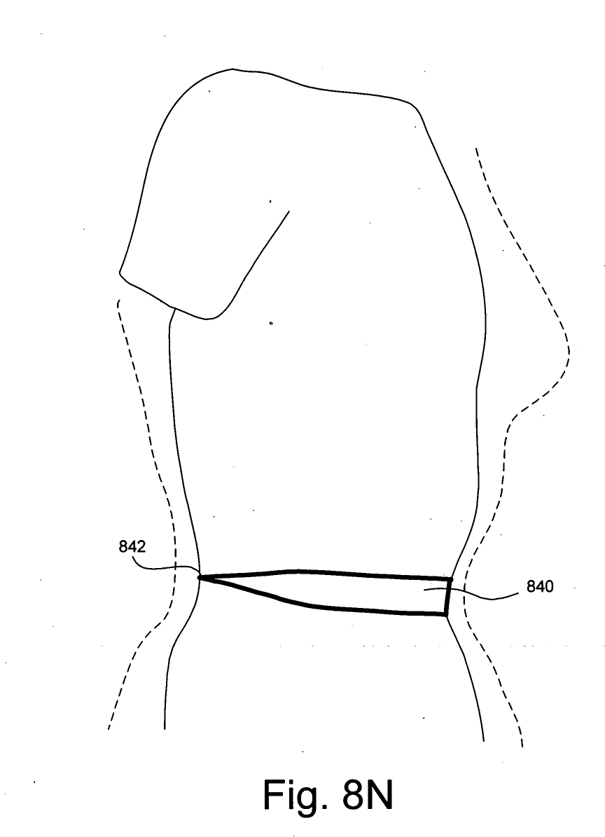

The ends 702, 704 of the band 700 can be configured to be fastened, tied, clipped, clamped and/or otherwise connected to each other to secure the band 700 to the body. Referring now to FIG. 8A, in one embodiment, the band 700 is a rectangular strip of material which does not include any fastening devices on the ends 702, 704. The ends 702, 704 of the band 700 in this embodiment can be connected by tying the ends 702, 704 together or stitching the ends 702, 704 together. In an embodiment, the band 700 can include marks 706 and/or numbers 708 to measure the circumference of the waist.

FIGS. 8B through 8Z illustrate various examples of fasteners, hooks, clips, adjusters and/or clamps which can be used to connect the ends 702, 704 of a band 700 together. FIG. 8B illustrates a band 700 having hooks 800 on the first end 702 and eyes 802 on the second end 704 of the band 700. FIG. 8C also illustrates a band 700 having hooks 804 on the first end 702 and eyes 806 on the second end 704 of the band 700. FIG. 8D illustrates a band 700 having a buckle 808 on the first end 702, wherein the second end 704 of the band 700 can be secured to the buckle 808. FIG. 8E illustrates a band 700 having a plurality of apertures 810 on the first end 702 and a loop 812 including fasteners 814 to fit inside the apertures 810 on the second end 704. FIG. 8F illustrates a band 700 having a loop 816 having a tightening device 818 on the first end 702, wherein the second end 704 of the band 700 can be secured to the first end 702 within the loop 816 using the tightening device 818. FIG. 8G illustrates a band 700 having a hinge 820, a band cover 822 and a tightening device 824 on the first end 702, wherein the second end 704 of the band 700 can be secured to the first end 702 under the band cover 822. FIG. 8H illustrates a band 700 having a crimper sleeve 826 to connect the first end 702 to the second end 704 of the band 700. FIG. 8I illustrates a band 700 having a stop sleeve 828 to connect the first end 702 to the second end 704 of the band 700. FIG. 8J illustrates a band 700 including a buckle 830 to connect the first end 702 to the second end 704 of the band 700. FIG. 8K illustrates a band 700 having a stop sleeve 832 to connect the first end 702 to the second end 704 of the band 700. FIG. 8L illustrates a band 700 including a clamp 834 on the first end 702 of the band 700 and built in striations 836 on the second end 704 of the band 700, wherein the second end 704 of the band 700 can be secured to the first end 702 by the clamp 834. FIG. 8M illustrates a band 700 including jack chain connectors 838 on both the first end 702 and the second end 704 of the band 700. FIG. 8N illustrates a band 700 wherein the front portion 840 of the band 700 is a rectangular strap and the rear portion 842 of the band 700 is a thin strap or wire 842. The strap 842 can easily be tightened or released. It is envisioned that the band 700 may include any other fastening devices that would be obvious to one skilled in the art without deviating from the scope of the invention.

While embodiments and applications of this invention have been shown and described, it would be apparent to those skilled in the art having the benefit of this disclosure, that many more modifications than mentioned above are possible without departing from the inventive concepts described herein.

1. A method of implanting an abdominal band subcutaneously for waistline reduction, which comprises:
making at least one incision along a waist, each incision extending through skin and fat, superficial to a fascia and muscle;
inserting a tool for dissection into the at least one incision;
developing a plane of dissection around the waist adjacent to the fascia and muscle with the tool for dissection, wherein the fat is separated from the fascia and muscle;
inserting the abdominal band into the plane of dissection;
wrapping the abdominal band around the waist within the plane of dissection, the abdominal band wrapped tight enough to reduce the size of the waist;
connecting the ends of the abdominal band; and
closing the at least one incision, wherein the abdominal band subcutaneously reduces the person's waistline.

2. The method of claim 1 wherein the step of making at least one incision includes making a peri-umbilical incision curved cephalad to the umbilicus.

3. The method of claim 1, wherein at least three incisions are made, one incision being posteriorly located and two opposed incisions being laterally located along the person's waistline.

4. The method of claim 1, wherein the step of developing a plane involves endoscopic dissection.

5. The method of claim 1, wherein the plane of dissection is developed using a hydro dissection tool.

6. The method of claim 1, wherein the plane of dissection is developed using a balloon dissection tool.

7. The method of claim 1, further comprising a step of inserting a curved probed into the plane of dissection to dilate the plane of dissection prior to inserting the abdominal band.

8. The method of claim 1, wherein the plane of dissection is developed below the umbilicus.

9. The method of claim 1, wherein the plane of dissection is developed above the umbilicus.

10. The method of claim 1, wherein the plane of dissection is developed both above and below the umbilicus.

11. The method of claim 1, wherein the ends of the band are connected by tying the ends of the band together.

12. The method of claim 1, wherein in the ends of the band are connected using a fastening device.

13. The method of claim 1, wherein the ends of the band include marks and numbers for measuring a circumference of the person's waist.

14. The method of claim 1, wherein the abdominal band is biocompatible, nonresorbable and nonbiodegradable.

15. The method of claim 1, wherein the abdominal band is elastic.

16. The method of claim 1, wherein the abdominal band is reversible.

17. The method of claim 1, wherein the at least one incision is 1 to 3 inches in length.

18. An abdominal band for subcutaneously reducing a person's waistline, comprising:
an elastic band adapted to be subcutaneously implanted around a person's abdomen;
said elastic band being 20 to 30 inches long, at least 1.25 inches wide and 1 to 3 mm thick; and
said elastic band being biocompatible, nonresorbable and nonbiodegradable.

19. An abdominal band for subcutaneously reducing a person's waistline, comprising:
a band adapted to be subcutaneously implanted around a person's abdomen;
said band being 20 to 50 inches long and at least 1 inch wide; and
said band being biocompatible, nonresorbable and nonbiodegradable.
